(19) United States
(12) Patent Application Publication
(10) Pub. No.: US 2024/0189519 A1
(43) Pub. Date: Jun. 13, 2024

(54) NEEDLE CAP FOR A MEDICAL SYRINGE

(71) Applicant: Becton, Dickinson and Company, Franklin Lakes, NJ (US)

(72) Inventors: Manish Kumar, Bengaluru (IN); Praveen Nalawade, Belgaum (IN); Shishir Prasad, Ramsey, NJ (US)

(73) Assignee: Becton, Dickinson and Company, Franklin Lakes, NJ (US)

(21) Appl. No.: 18/080,428

(22) Filed: Dec. 13, 2022

(51) Int. Cl. A61M 5/32; A61B 5/15

(52) U.S. Cl. CPC A61M 5/3202; A61B 5/150603

(57) ABSTRACT

A needle cap for a medical syringes facilitates aspiration of medicine into the syringe, through the stopper of a medicine vial, directly through the injection needle of the syringe. A proximal end of the needle cap is coupled to a needle hub of the syringe, with an outer diameter of the syringe needle captured within and in sealing engagement with an internal diameter of a needle seal. An open tip tapered distal end of the needle cap is inserted through the stopper of the medicine vial. Retraction of the syringe plunger aspirates medicine through the open tip of the needle cap, directly through the syringe needle, into the syringe chamber. Upon completion of medicine aspiration into the syringe, the needle cap is removed, and the syringe is ready for injection of medicine to the patient.

### TECHNICAL FIELD

The present disclosure generally relates to needle caps configured for capping medical injection syringes. More particularly, embodiments of needle caps of the present disclosure facilitate direct aspiration of medicine from a vial into an injection needle of an injection medical syringe, while the needle cap is coupled to the needle hub of the syringe.

### BACKGROUND

Liquid drugs and other pharmaceuticals are typically stored in glass ampules or vials and sealed with elastomer stoppers, including by way of non-limiting example stoppers with or without slit septums. In order to aspirate the drug contained within the vial to a syringe for subsequent administration to a patient, cylindrical exterior profile, blunt fill needles are typically affixed to a Luer connector of the syringe, in order to pierce the stopper/septum, followed by retraction of the syringe plunger to aspirate the drug into the syringe chamber. After filling the syringe with a desired quantity of the drug, the blunt fill needle is then detached from the syringe and replaced with a hypodermic injection needle for injection into the patient. Attachment and replacement of two separate types of needles to the syringe is cumbersome and time consuming. Each separate attachment introduces potential drug administration errors and/or contamination. When the blunt fill needle is separated from the syringe any remaining drug aspirated into the fill needle internal cavity that is not contained within the syringe cavity is wasted. Generally cylindrical profile blunt fill needles do not allow complete aspiration of all of the drug contained within the vial because the flat needle tip cannot remain in contact with residual drug contained within the bottom corners of the vial.

### SUMMARY

Embodiments of the disclosure pertain to a needle cap configured for capping medical syringes that facilitates aspiration of medicine into the syringe, through the stopper of a medicine vial. Medicine is aspirated from the vial, directly through the injection needle of the syringe, while the needle cap is coupled to the needle hub of the syringe. A proximal end of the needle cap is coupled to a needle hub of the syringe, with an outer diameter of the syringe needle captured within and in sealing engagement with an internal diameter of a needle seal. An open tip tapered distal end of the needle cap is inserted through the stopper of the medicine vial. Retraction of the syringe plunger aspirates medicine through the open tip of the needle cap, directly through the syringe needle, into the syringe chamber. Upon completion of medicine aspiration into the syringe, the needle cap is removed, and the syringe is ready for administration of medicine to the patient.

One aspect of the present disclosure pertains to a needle cap for a medical syringe. The needle cap includes a tubular body, having an inner wall surface defining a cap internal cavity. The inner wall surface defines an open-ended, female Luer-taper profile, first cavity portion on a proximal end of the tubular body that is configured to be selectively coupled to a needle hub of a syringe. The inner wall surface defines an open cap tip on a distal end of the tubular body. A needle seal within the cap internal cavity, divides the cap internal cavity into second proximal and third distal portions that are isolated from each other. The needle seal defines a through-bore that is in communication with both of the isolated second proximal and third distal portions of the cap internal cavity. The through-bore is in unobstructed, direct fluid communication with the open cap tip, and said it defines an internal diameter configured for mated sealing engagement with an outer diameter of a syringe needle when said needle is captured therein. The tubular body has a tapered, frusto-conical outer surface profile on the distal end thereof, which terminates in the open cap tip.

Another aspect of the present disclosure pertains to a syringe with a needle cap. The syringe comprises a syringe barrel having proximal and distal ends and a syringe cavity therein, along with a plunger and coupled plunger stopper in the proximal end of the syringe barrel. A male Luer connector is oriented on the proximal end of the syringe barrel, in fluid communication with the syringe cavity. A Luer needle hub is coupled to the male Luer connector, in fluid communication with the syringe cavity. The syringe includes a hollow syringe needle having a proximal end coupled to the Luer needle hub, in fluid communication with the syringe cavity, and a distal open needle tip. The syringe includes a needle cap having a tubular body with an inner wall surface defining a cap internal cavity therein, which captures the distal open needle tip. The inner wall surface of the needle cap defines an open-ended, female Luer-taper profile, first cavity portion on a proximal end of the tubular body that is coupled to the needle hub. The internal wall surface of the cap also defines an open cap tip on a distal end of the tubular body. A needle seal within the cap internal cavity, divides the cap internal cavity into second proximal and third distal portions that are isolated from each other. The needle seal defines a through-bore in communication with both of the isolated second proximal and third distal portions, The through-bore has an internal diameter configured for mated sealing engagement with an outer diameter of the syringe needle captured therein. The distal open needle tip is oriented in the third distal portion of the cap internal cavity in unobstructed, direct fluid communication with the open cap tip. The tubular body has a tapered, frusto-conical outer surface profile on the distal end thereof, which terminates in the open cap tip.

### BRIEF DESCRIPTION OF THE DRAWINGS

Exemplary embodiments of the disclosure are further described in the following detailed description in conjunction with the accompanying drawings, in which:

FIG. 1 is an isometric view of a medical syringe with a coupled needle cap and needle shield of the present disclosure;

FIG. 2 is an axial cross-sectional view of the medical syringe with a coupled needle cap and needle shield of FIG. 1;

FIG. 3A is an axial cross-sectional view of the needle cap with an inserted syringe needle and needle hub of FIG. 2;

FIG. 3B is an axial cross-sectional view of an alternative embodiment of a medical syringe with a couple needle cap and a needle shiel with an inserted syringe needle and needle hub;

FIG. 4 is an exploded view of the medical syringe, needle cap and needle shield of FIG. 1; and

FIG. 5 is a view of the needle cap and syringe of FIG. 1 aspirating medicine through a stopper of a medicine vial.

To facilitate understanding, identical reference numerals have been used, where possible, to designate identical elements that are common to the figures. The figures are not drawn to scale.

### DETAILED DESCRIPTION

A needle cap for a medical syringes facilitates aspiration of medicine into the syringe, through the stopper of a medicine vial. Medicine is aspirated from the vial, directly through the injection needle of the syringe, while the needle cap is coupled to the needle hub of the syringe. A proximal end of the needle cap is coupled to a needle hub of the syringe, with an outer diameter of the syringe needle captured within and in sealing engagement with an internal diameter of a needle seal. An open tip tapered distal end of the needle cap is inserted through the stopper of the medicine vial. Retraction of the syringe plunger aspirates medicine through the open tip of the needle cap, directly through the syringe needle, into the syringe chamber. The needle seal blocks fluid communication with a proximal portion of the needle cap cavity between the seal and the proximal end of the needle cap, so that excess medicine that was not aspirated into the syringe chamber is not wasted. The tapered distal tip of the needle cap facilitates complete aspiration of medicine from the medicine vial. Upon completion of medicine aspiration into the syringe, the needle cap is removed, and the syringe is ready for administration of medicine to the patient.

Before describing several exemplary embodiments of the disclosure, it is to be understood that the disclosure is not limited to the details of construction or process steps set forth in the following description. The disclosure is capable of other embodiments and of being practiced or being conducted in many ways.

In this disclosure, a convention is followed wherein the distal end of the device is the end closest to a patient and the proximal end of the device is the end away from the patient and closest to a practitioner.

With respect to terms used in this disclosure, the following definitions are provided. As used herein, the use of “a,” “an,” and “the” includes the singular and plural.

As used herein, the term “Luer connector” refers to a connection collar that is the standard way of attaching syringes, catheters, hubbed needles, IV tubes, etc. to each other. In some embodiments, the Luer connector comprises a luer slip connector including male and female interlocking tubes (a male luer slip connector and a female luer slip connector), slightly tapered to hold together better with even just a simple pressure/twist fit. Luer connectors can optionally include an additional outer rim of threading, allowing them to be more secure. The Luer connector male end is generally associated with a flush syringe and can interlock and connect to the female end located on the vascular access device (VAD) Luer connector also has a distal end channel that releasably attaches the Luer connector to the hub of a VAD, and a proximal end channel that releasably attaches the Luer connector to the barrel of a syringe.

As used herein, ISO 80369-7:2016 defines a specification for standard Luer connectors including a 6% taper between the distal end and the proximal end. A male standard luer connector increases from the open distal end to the proximal end. A female standard luer connector decreases from the open proximal end to the distal end. According to ISO 80369-7:2016, a male standard luer connector has an outer cross-sectional diameter measured 0.75 mm from the distal end of the tip of between 3.970 mm and 4.072 mm. The length of the male standard luer taper is between 7.500 mm to 10.500 mm. The outer cross-sectional diameter measured 7.500 mm from the distal end of the tip is between 4.376 mm and 4.476 mm. As used herein, the phrases “male standard luer connector” and “female standard luer connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “slope”, “wall”, “top”, “side”, “bottom” and others are used throughout this specification to facilitate understanding, it is not intended to limit any components that can be used in combinations or individually to implement various aspects of the embodiments of the present disclosure.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

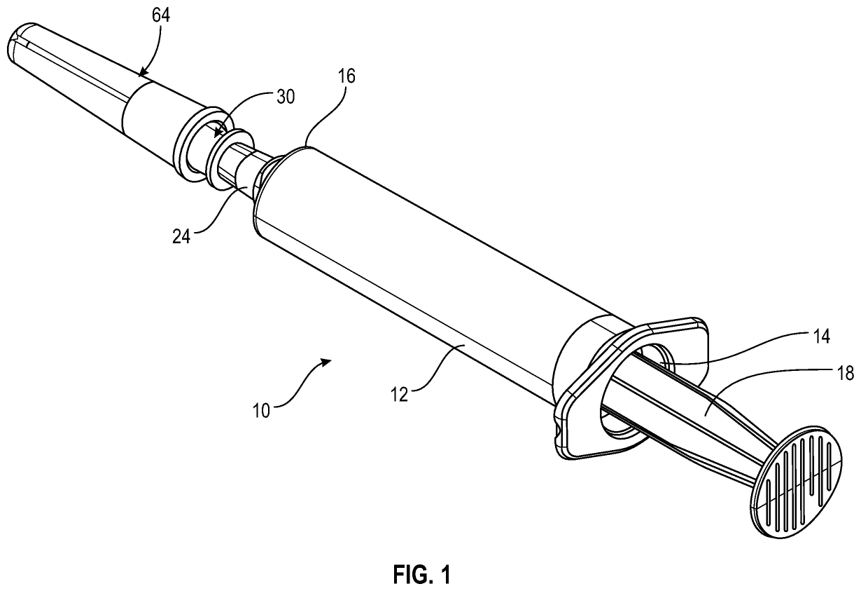

In an exemplary implementation of the embodiments of present disclosure, FIGS. 1-3 depict a syringe 10. The syringe 10 includes a syringe barrel 12, having proximal end 14 and distal end 16. A plunger 18 and a coupled plunger stopper 20 are inserted in the proximal end 14 of the syringe barrel 12. A syringe cavity 21 is defined between the distal end 16 of the syringe barrel 12 and a distal end of the plunger stopper 20. A male Luer connector 22 on the distal end 16 of the syringe barrel 12 is in fluid communication with the syringe cavity 21. A needle hub 24 is coupled to the male Luer connector 22, which is also in fluid communication with the syringe cavity 21. A hollow syringe needle 26 has a proximal end coupled to the needle hub 24 and a sharpened, distal open needle tip 28 for subcutaneous puncture of a patient. The distal open needle tip 28 is in fluid communication with the syringe cavity 21 via the lumen defined within the syringe needle 26.

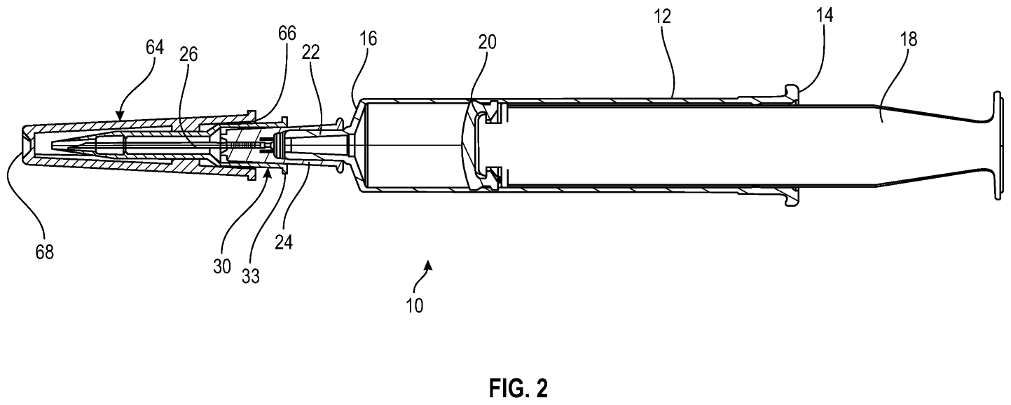

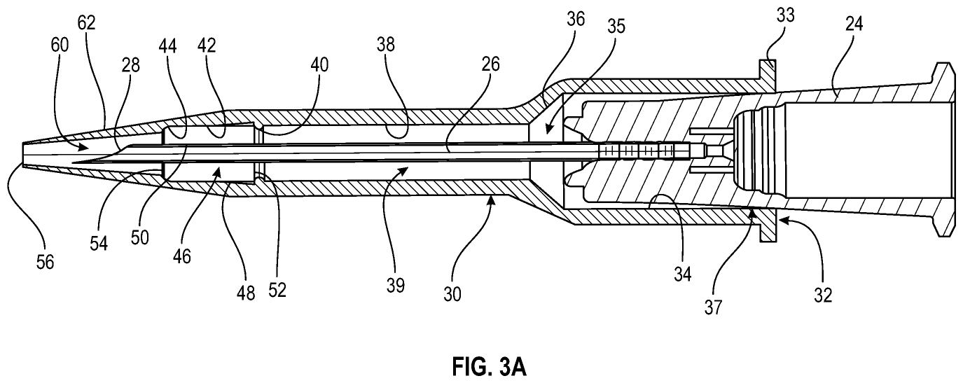

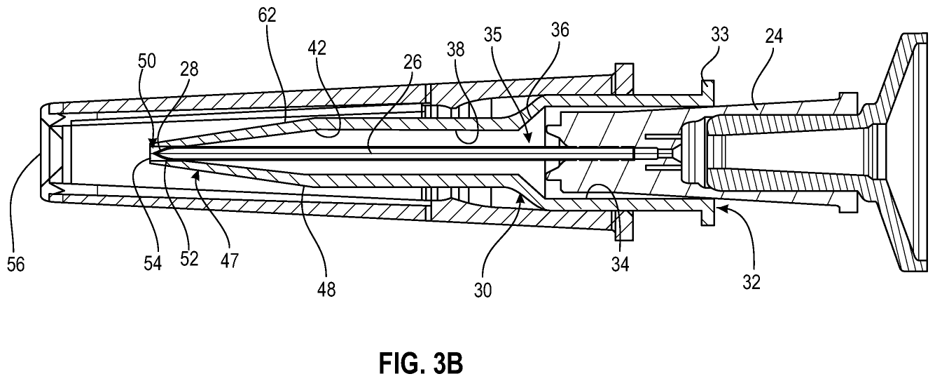

Referring to FIGS. 2 and 3, a needle cap 30 has a hollow tubular body 31, with a proximal end 32 that incorporates a radially outwardly projecting gripping flange 33. A first inner wall surface 34 of the hollow tubular body 31 defines an open-ended, female Luer-taper profile, first cavity portion 35. The first inner wall surface 34 is coupled to the exterior surface of the needle hub 24 in mutual slip-fit, friction engagement. In other embodiments, the first inner wall surface defines a female threaded luer connector comprising internal female threads of a threaded Luer connector that are configured for engagement with corresponding male threads of a threaded Luer hub comprising a male threaded Luer connector. The inner wall of the hollow tubular body 31 defines a necked portion 36 distal the first inner wall surface 34. The distal end of the necked portion 36 within the hollow tubular body 31 has a reduced diameter inner wall surface 38, which in turn defines a proximal second portion 39 of the needle cavity and an inwardly-projecting radial lip 40. The inner wall of the hollow tubular body 31 distal the radially projecting lip 40 further defines a seal cavity inner wall 42 and a seal retention shoulder 44.

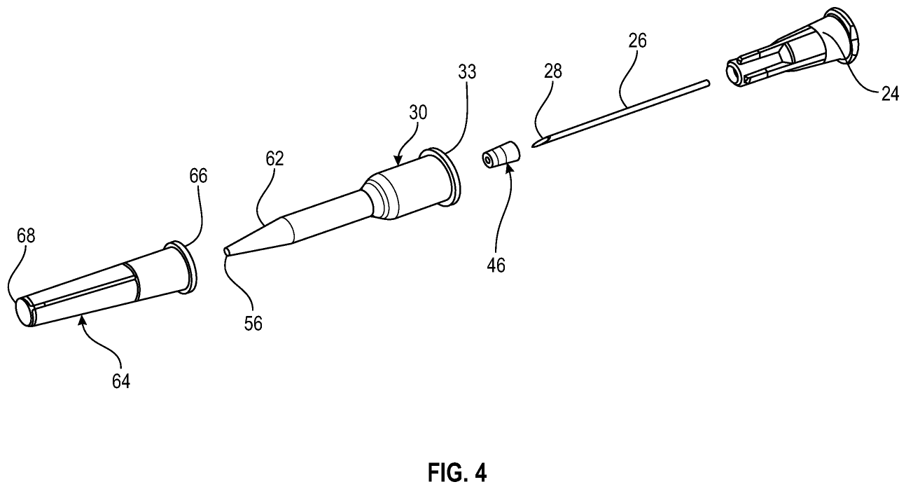

An annular needle seal 46 is oriented within the seal cavity inner wall 42 between the inwardly-projecting radial lip 40 and the seal retention shoulder 44. The needle seal 46 and has an outer diameter surface 48 in sealing engagement with the seal cavity inner wall 42. The annular needle seal defines a through-bore 50 between its proximal axial end 52 and its distal axial end 54. The through-bore 50 receives and captures the syringe needle 26 therein and has an inner diameter surface in sealing engagement with the outer surface of the syringe needle. The open needle tip 28 projects outwardly from the distal axial end 54 of the needle seal 46 and is in fluid communication with an open distal cap tip 56 of the needle cap 30. The open distal cap tip 56 of the needle cap 30 is in direct, unobstructed fluid communication with syringe cavity 21, via a fluid flow path through the lumen of the syringe needle 26 from its open needle tip 28 through the needle hub 24. The needle seal 46 separates the internal cavity of the needle cap 30 into the proximal second portion 39, defined between its proximal axial end 52 and the first cavity portion 35 and a distal third portion 60 defined between its distal axial end 54 and the open distal cap tip 56 of the needle cap. In the syringe embodiment of FIGS. 1-4, the needle seal 46 is a separate component comprising an elastomeric material. In other embodiments the needle seal is integrally formed within the needle cap.

Medicine aspirated through the open distal cap tip 56 only enters the distal third portion 60 of the needle cap 30 and directly flows in unobstructed fashion through the open needle tip 28 and ultimately into the syringe barrel 12 of the syringe 10. The needle seal 46, in scaling engagement with seal cavity inner wall 42 and the outer diameter of the syringe needle 26, the blocks fluid leakage into the proximal second portion 39 and the first cavity portion 35 of the needle cap 30, so that aspirated medicine is not trapped and wasted within the needle cap. In some embodiments, so-called “dead space” volume within the third distal portion 60 of the needle cap 30 is minimized, in order to minimize wasted aspirated medicine.

Referring to FIGS. 3A-B and 4, the outer exterior surface profile of the needle cap 30 forms a tapered, distal end 62, which terminates in the open distal cap tip 56. In other embodiments, the tapered distal end comprises other tapered polygonal profiles that terminate in the open distal cap tip. FIG. 3B shows an alternative embodiment in which the annular needle seal 46 is eliminated, and the needle seal is integrally formed within the needle cap 30. In this alternative embodiment, the connection between needle hub and needle cap is airtight, eliminating the additional annular needle seal 46 element between the needle cap 30 and the syringe needle 26. The distal open needle tip 28 is positioned proximal to the distal cap tip 56 to avoid accidental needle stick injury and at the same time minimizing drug wastage during aspiration from a medical container, for example, a vial. Any such drug wastage is expected to be lower than using a separate blunt fill needle for aspiration of drug. As shown in FIG. 3B, the distal seal end 52 is in contact with syringe needle 26 adjacent to the open distal needle tip, and an integral annular needle seal 47 is provided, not by the separate component shown in FIG. 3A, but between the distal seal end 52 and the proximal seal end 54, thereby eliminating the proximal second portion 39 and distal seal portion 60 in the embodiment shown in FIG. 3A. In the embodiment in FIG. 3B, the needle seal is integrally formed with the cap, which forms the mated scaling engagement with an outer diameter of a syringe needle when said needle is captured therein. In other embodiments, the annular needle seal 47 in FIG. 3B and the additional annular needle seal 46 element in FIG. 3A can be supplemented or replaced with a tight annular seal at location formed at the interface 37 of first inner wall surface 34 and the exterior surface of the needle hub 24 in mutual slip-fit, friction engagement. Thus, a tight annular seal at the interface 37 in some embodiments, would eliminate the need for annular needle seal 47 or the additional annular needle seal 46 element. If a tight seal is formed at interface 37 air will not be drawn in from this interface and liquid would not leak at the needle cap-needle hub interface.

A selectively removeable needle shield 64 is coupled to and covers the distal end of the needle cap 30 is shown in FIGS. 1, 2 and 4. The needle shield 64 has a proximal open end 66 and a blunt, closed tip 68 on its distal end. The needle shield 64 seals the open distal cap tip 56 to prevent entry of foreign matter into the needle cap 30 and the syringe 10; its blunt tip 68 also prevents inadvertent piercing of sterile syringe packaging that might otherwise occur if the tapered distal end 62 of the syringe was not covered by the needle shield.

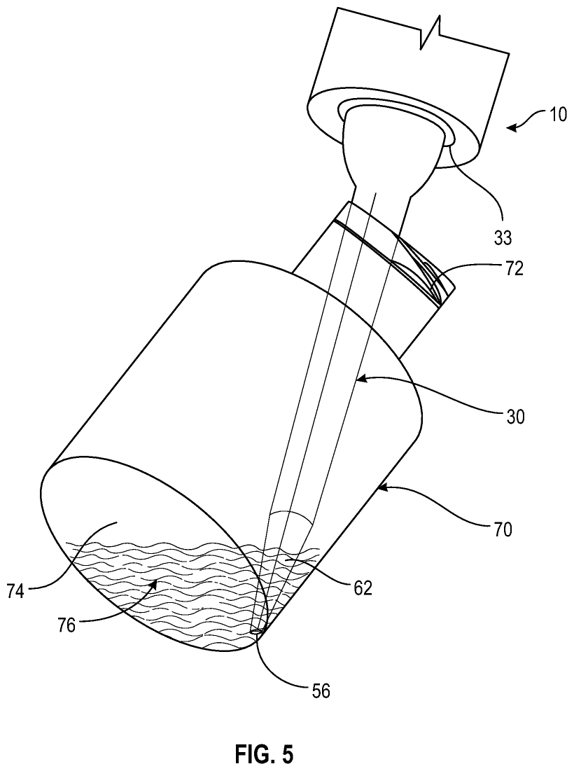

As shown in FIG. 5, the tapered distal end 62 of the tip of the needle cap 30 facilitates direct aspiration of medicine from a drug vial 70 while the syringe needle remains in situ within the needle cap. The tapered distal end 62 of the needle cap 30 pierces a septum 72 of the drug vial 70 and reaches the vial bottom 74, to aspirate the medicine 76 or other medical liquid, such as water for injection or saline flushing solution, directly into the syringe 10. The tapered distal end 62 of the needle cap 30 allows its open cap tip 56 to extract all medicine from the vial bottom 74. The smooth, tapered distal end 62 of the needle cap 30 reduces potential infiltration of damaged vial septum 72 particulate matter that might otherwise contaminate the medicine 76 if a sharpened syringe needle tip were used directly to aspirate medicine from the vial 70. Beneficially, while the needle cap 30 covers the syringe needle, it also protects the syringe needle from bending or needle tip glinting contact damage. After aspirating the desired quantity of drug into the syringe chamber, a medical practitioner removes the needle cap 30 to expose the injection needle for subsequent injection into the patient. Handling of exposed, sharp injection needles is minimized because the needle and needle hub remain affixed to the syringe during medicine aspiration into the syringe.

In contrast, without the benefit of use of the needle cap 30 of this disclosure, a medical practitioner exercising patient drug administration protocols would otherwise need to affix a blunt fill needle to a naked syringe Luer connector, by first removing its associated injection needle or by utilizing a syringe without an injection needle. After blunt fill needle affixation, the medical practitioner aspirates medicine from a vial into the syringe chamber, and thereafter replaces the blunt fill needle with an injection needle, in order to administer the drug to the patient. Use of a separate, known blunt fill needle, rather than the needle cap 30 of the present disclosure, requires additional syringe preparation steps, thereby increasing possibility of administration error and potential sterility compromise.

The syringe and syringe needle cap embodiments disclosed herein are constructed from medical grade materials known to one skilled in the art. In some embodiments, described barrels, plungers and shafts are fabricated with but not limited to polypropylene and polycarbonate polymers. In some embodiments, seals are fabricated with elastomers, such as by way of non-limiting example, cross-linked rubber, thermoplastic elastomers, or fiber-filled polytetrafluoroethylene (PTFE) polymers. In some embodiments, stoppers are fabricated with polyisoprene polymers or other known elastomers such as butyl rubber, thermoplastic elastomer or thermoplastic vulcanizates.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. A needle cap for a medical syringe, comprising:
a tubular body having an inner wall surface defining a cap internal cavity therein, the inner wall surface further defining an open-ended, female Luer-taper profile, first cavity portion on a proximal end of the tubular body that is configured to be selectively coupled to a needle hub of a syringe, and the inner wall surface defining an open cap tip on a distal end of the tubular body;
a needle seal within the cap internal cavity, dividing the cap internal cavity into a second proximal portion and a third distal portion that are isolated from each other, the needle seal defining a through-bore in communication with both of the second proximal portion and the third distal portion of the cap internal cavity, the through-bore in unobstructed, direct fluid communication with the open cap tip, and the through-bore having an internal diameter configured for mated sealing engagement with an outer diameter of a syringe needle when the needle is captured therein; and
the tubular body having a tapered, frusto-conical outer surface profile on the distal end thereof, which terminates in the open cap tip.

2. The needle cap of claim 1, the inner wall surface of the tubular body defining radially inwardly directed lip abutting a proximal axial end of the needle seal and a retention shoulder abutting a distal axial end of the needle seal.

3. The needle cap of claim 2, further comprising a separate elastomer needle seal.

4. The needle cap of claim 1, wherein the needle seal is integrally formed with the cap, which forms the mated sealing engagement with an outer diameter of a syringe needle when said needle is captured therein.

5. The needle cap of claim 2, the tubular body defining a reduced diameter, necked portion intermediate the female Luer-taper profile, first cavity portion and the tapered, frusto-conical distal end thereof, the needle seal oriented distal said necked portion.

6. The needle cap of claim 1, the tubular body defining a reduced diameter, necked portion intermediate the female Luer-taper profile, first cavity portion and the tapered, frusto-conical distal end thereof, the needle seal oriented distal said necked portion.

7. The needle cap of claim 1, the proximal end of the tubular body defining an outwardly projecting gripping flange.

8. The needle cap of claim 1, the inner wall surface defining the first cavity portion of the cap internal cavity further comprising a female Luer slip connector.

9. The needle cap of claim 1, the inner wall surface defining the first cavity portion of the cap internal cavity further comprising a female threaded Luer connector.

10. The needle cap of claim 1, further comprising an elastomer needle seal.

11. The needle cap of claim 1, further comprising a needle shield coupled to the distal end of the tubular body and sealing the open cap tip.

12. A syringe comprising:
a syringe barrel having proximal and distal ends and a syringe cavity therein;
a plunger and coupled plunger stopper in the proximal end of the syringe barrel;
a male Luer connector on the proximal end of the syringe barrel, in fluid communication with the syringe cavity;
a Luer needle hub coupled to the male Luer connector, in fluid communication with the syringe cavity;
a hollow syringe needle having a proximal end coupled to the Luer needle hub, in fluid communication with the syringe cavity, and a distal open needle tip;
a needle cap having a tubular body with an inner wall surface defining a cap internal cavity therein, which captures the distal open needle tip, the inner wall surface further defining an open-ended, female Luer-taper profile, first cavity portion on a proximal end of the tubular body that is coupled to the needle hub, and the inner wall surface defining an open cap tip on a distal end of the tubular body;
a needle seal within the cap internal cavity, dividing the cap internal cavity into a second proximal portion and a third distal portion, which are isolated from each other, the needle seal defining a through-bore in communication with both of the second proximal and the third distal portions, the through-bore having an internal diameter configured for mated sealing engagement with an outer diameter of the syringe needle captured therein;
the distal open needle tip oriented in the third distal portion of the cap internal cavity in unobstructed, direct fluid communication with the open cap tip; and
the tubular body having a tapered, frusto-conical outer surface profile on the distal end thereof, which terminates in the open cap tip.

13. The syringe of claim 12, the internal wall surface of the tubular body defining radially inwardly-projecting lip abutting a proximal axial end of the needle seal and a retention shoulder abutting a distal axial end of the needle seal.

14. The syringe of claim 13, further comprising an elastomer needle seal.

15. The syringe of claim 13, the tubular body defining a reduced diameter, necked portion intermediate the female Luer-taper profile, cavity portion and the tapered, frusto-conical distal end thereof, the needle seal oriented distal said necked portion.

16. The syringe of claim 12, the tubular body defining a reduced diameter, necked portion intermediate the female Luer-taper profile, cavity portion and the tapered, frusto-conical distal end thereof, the needle seal oriented distal said necked portion.

17. The syringe of claim 12, the proximal end of the tubular body defining an outwardly projecting gripping flange.

18. The syringe of claim 12, the inner wall surface defining the first cavity portion of the cap internal cavity further comprising a female, Luer slip connector, mating with a corresponding male, Luer slip connector of the needle hub.

19. The syringe of claim 12, the inner wall surface defining the first cavity portion of the cap internal cavity further comprising a female, threaded Luer connector, mating with a corresponding male, threaded Luer connector of the needle hub.

20. The syringe of claim 12, further comprising a needle shield coupled to the distal end of the tubular body and sealing the open cap tip.
